(19) United States
(12) Patent Application Publication
(10) Pub. No.: US 2024/0185509 A1
(43) Pub. Date: Jun. 6, 2024

(54) 3D RECONSTRUCTION OF ANATOMICAL IMAGES

(71) Applicant: RSIP Vision Ltd., Jerusalem (IL)

(72) Inventors: Ilya Kovler, Jerusalem (IL); Daniel Doktofsky, Jerusalem (IL); Barel Levy, Jerusalem (IL); Hamza Abudayyeh, Jerusalem (IL); Moshe Safran, Jerusalem (IL)

(21) Appl. No.: 18/548,578

(22) PCT Filed: Apr. 25, 2022
(86) PCT No.: PCT/IB2022/053810

§ 371 Date: Sep. 1, 2023

Related U.S. Application Data
(60) Provisional application No. 63/179,485, filed on Apr. 25, 2021.

(51) Int. Cl. G06T 15/08; G06T 7/11

(52) U.S. Cl. CPC G06T 15/08; G06T 7/11; G06T 2207/10081; G06T 2207/10088; G06T 2207/10104; G06T 2207/20081; G06T 2207/20084; G06T 2207/30008; G06T 2210/41

(57) ABSTRACT

A computer-implemented method of using a neural network to reconstruct three dimensional (3D) images of an anatomical structure from two-dimensional (2D) images including the steps of: (a) in a 3D image training dataset associated with the anatomical structure to be reconstructed, creating respective 3D ground truth masks and/or heat maps for each 3D training image; (b) obtaining 2D images of the anatomical structure and their associated calibration parameters; (c) generating a 3D volume including a 3D array of voxels and a plurality of channels, at least some of the channels associated with the one or more generated 2D images and associated calibration parameters of step (b); and (d) training a neural network by associating the 3D ground truth masks and/or heat maps with the generated 3D volume.

### CROSS-REFERENCE TO RELATED APPLICATIONS

This is a 371 application from international patent application No. PCT/IB2022/053810 filed Apr. 25, 2022, which claims priority from U.S. Provisional Patent Application No. 63/179,485 filed Apr. 25, 2021, and which is expressly incorporated herein by reference in its entirety.

### FIELD

The subject matter disclosed herein relates in general to reconstruction of three-dimensional (3D) anatomical images, and in particular to 3D reconstruction of anatomical images from two-dimensional (2D) anatomical images.

### BACKGROUND

Image based planning is an important step in many medical procedures including orthopaedic surgery such as joint replacement. Detailed 3-dimensional (3D) understanding of the patient anatomy can have a major contribution to the accuracy of the procedure and to patient outcomes. For example, in hip replacement surgery, careful selection and positioning of the implant is vital. In hip arthroscopy procedures, a full understanding of the detailed patient anatomy is instrumental in enabling the physician to precisely plan interventions such as bone resection. A 3D model may be obtained from CT or MRI imaging which typically require relatively high levels of ionizing radiation (CT) and may involve high costs (especially for MRI). These methods may also be limited by low image resolution. In the current standard of care, many patients undergo only 2D imaging such as X-ray or fluoroscopy which may yield only partial information.

### SUMMARY

In various embodiments there is a computer-implemented method of using a neural network to reconstruct three dimensional (3D) images of an anatomical structure from two-dimensional (2D) images including the steps of: (a) in a 3D image training dataset associated with the anatomical structure to be reconstructed, creating respective 3D ground truth masks and/or heat maps for each 3D training image; (b) obtaining 2D images of the anatomical structure and their associated calibration parameters; (c) generating a 3D volume comprising a 3D array of voxels and a plurality of channels, at least some of the channels associated with the one or more generated 2D images and associated calibration parameters of step (b); and (d) training a neural network by associating the 3D ground truth masks and/or heat maps with the generated 3D volume.

In some embodiments, the method further includes, between steps (a) and (b), changing a modality of one or more 3D images in the 3D image training dataset using a 3D style transfer algorithm.

In some embodiments, the method further includes, between steps (a) and (b), augmenting features in one or more 3D training images using an augmentation algorithm.

In some embodiments, the method further includes, between steps (b) and (c), changing a modality of one or more of the 2D images using a 2D style transfer algorithm.

In some embodiments, the method further includes, between steps (b) and (c), augmenting features in one or more of the 2D images using an augmentation algorithm.

In some embodiments, the method further includes generating 2D masks from the 2D images and/or the 3D ground truth masks.

In some embodiments, the method further includes projecting the 2D masks into the plurality of channels in the 3D volume considering the associated calibration parameters.

In some embodiments, the 2D images include at least one or more digitalized reconstruction radiographic (DRR) images, and the associated calibration parameters are generated considering the expected calibration parameters of real-world inputs.

In some embodiments, the method further includes, after step (d), repeating steps (a) to (d) one or more times to determine an optimal 2D image view angle relative to the training anatomical structure.

In some embodiments, the method further includes, using the trained neural network of step (d), the steps of: (e) obtaining a plurality of 2D images of the anatomical structure; (f) determining calibration parameters and alignment parameters for the plurality of 2D images of step (e); (g) generating a 3D volume comprising a plurality of channels, at least some of the channels associated with the plurality of 2D images of step (e); and (h) reconstructing 3D masks and/or heat maps from the generated 3D volume of step (g) by applying the trained neural network of step (d).

In some embodiments, the method further includes generating meshes and/or landmark coordinates.

In some embodiments, the method further includes detecting a calibration jig on a plurality of 2D images acquired from a subject, each 2D image comprising a view angle included in the calibration parameters of step (f).

In some embodiments, the method further includes identifying locations on the calibration jig and assigning coordinates to the locations.

In some embodiments, the method further includes changing a modality of one or more of the 2D images acquired of the subject using a 2D style transfer algorithm.

In some embodiments, the method further includes identifying anatomical landmarks in one or more of the 2D images acquired of the subject using a landmark detection algorithm

In some embodiments, the method further includes performing 2D image segmentation in one or more of the 2D images acquired of the subject.

In some embodiments, the method further includes projecting 2D masks into the 3D volume considering the calibration parameters determined in step (f).

In some embodiments, the method further includes, after step (h), repeating steps (e) to (h) one or more times to generate multiple reconstruction of the anatomical structure to be reconstructed and/or other anatomical structures in the subject.

In various embodiments there is system for reconstructing 3D images of an anatomical structure from 2D images including a storage device including a 3D training dataset; one or more processors; and one or more memories storing instructions executable by the one or more processors and which cause the system to perform the following steps: (a) creating respective 3D ground truth masks and/or heat maps for each 3D training image in the 3D image training dataset associated with the anatomical structure to be reconstructed; (b) obtaining 2D images of the anatomical structure and their associated calibration parameters; (c) generating a 3D volume comprising a 3D array of voxels and a plurality of channels, at least some of the channels associated with the one or more generated 2D images and associated calibration parameters of step (b); and (d) training a neural network by associating the 3D ground truth masks and/or heat maps with the generated 3D volume.

In some embodiments, the system wherein, between steps (a) and (b), a modality of one or more 3D images in the 3D image training dataset is changed using a 3D style transfer algorithm.

In some embodiments, the system wherein, between steps (a) and (b), features in one or more 3D images in the 3D image training dataset are augmented using an augmentation algorithm.

In some embodiments, the system wherein, between steps (b) and (c), a modality of one or more of the 2D images is changed using a 2D style transfer algorithm.

In some embodiments, the system wherein, between steps (b) and (c), features in one or more of the 2D DRR images are augmented using an augmentation algorithm.

In some embodiments, the system further including generating 2D masks from the 2D images and/or the 3D ground truth masks.

In some embodiments, the system further including projecting the 2D masks into the plurality of channels in the 3D volume considering the associated calibration parameters.

In some embodiments, the system wherein the 2D images include at least one or more digitalized reconstruction radiographic (DRR) images, and the associated calibration parameters are generated considering the expected calibration parameters of real-world inputs.

In some embodiments, the system wherein, after step (d), repeating steps (a) to (d) one or more times to determine an optimal 2D image view angle relative to the training anatomical structure.

In some embodiments, the system further includes, using the trained neural network of step (d), performing the steps of: (e) obtaining a plurality of 2D images of the anatomical structure; (f) identifying locations on the calibration jig and assigning coordinates to the locations; (f) determining calibration parameters and alignment parameters for the plurality of 2D images of step (e); (g) generating a 3D volume comprising a plurality of channels, at least some of the channels associated with the plurality of 2D images of step (e); and (h) reconstructing 3D masks and/or heat maps of from the generated 3D volume of step (g) by applying the trained neural network of step (d).

In some embodiments, the system further includes changing a modality of one or more of the 2D images acquired of the subject using a 2D style transfer algorithm.

In some embodiments, the system further includes generating meshes and/or landmark coordinates.

In some embodiments, the system further includes detecting a calibration jig on a plurality of 2D images acquired from a subject, each 2D image comprising a view angle included in the calibration parameters of step (f).

In some embodiments, the system further includes identifying locations on the calibration jig and assigning coordinates to the locations.

In some embodiments, the system further includes changing a modality of one or more of the 2D images acquired of the subject using a 2D style transfer algorithm.

In some embodiments, the system further includes identifying anatomical landmarks in one or more of the 2D images acquired of the subject using a landmark detection algorithm

In some embodiments, the system further includes performing 2D image segmentation in one or more of the 2D images acquired of the subject.

In some embodiments, the system further includes projecting 2D masks into the 3D volume considering the calibration parameters determined in step (f).

In some embodiments, the system further includes, after step (h), repeating steps (e) to (h) one or more times to generate multiple reconstruction of the anatomical structure to be reconstructed and/or other anatomical structures in the subject.

### BRIEF DESCRIPTION OF THE DRAWINGS

Non-limiting examples of embodiments disclosed herein are described below with reference to figures attached hereto that are listed following this paragraph. The drawings and descriptions are meant to illuminate and clarify embodiments disclosed herein and should not be considered limiting in any way. Like elements in different drawings may be indicated by like numerals. Elements in the drawings are not necessarily drawn to scale. In the drawings:

FIG. 1 which schematically illustrates a block diagram of an exemplary system for AI-based, 3D reconstruction of anatomical images from two-dimensional 2D anatomical images using neural networks;

FIG. 2 illustrates an exemplary flow chart of a training operation of a training neural network the system of FIG. 1;

FIG. 3 illustrates an exemplary flow chart of a 3D anatomical reconstruction operation using a reconstruction neural network in the system of FIG. 1;

FIG. 4A shows an exemplary DRR of a hip joint viewed from an anterior-posterior angle, as generated using the training neural network of FIG. 3;

FIG. 4B shows an exemplary DRR of the hip joint of FIG. 4A viewed from a +45 degrees angle, as generated using the training neural network of FIG. 3;

FIG. 4C shows an exemplary DRR of the hip joint of FIG. 4A viewed from a −45 degrees angle, as generated using the training neural network of FIGS. 3; and

FIG. 5 shows an exemplary 3D reconstruction output volume including a representation of mesh models of a portion of a pelvis, a femur, and the joint joining the pelvis and the femur.

### DETAILED DESCRIPTION

Previous approaches to 2D-to-3D reconstruction for orthopaedic surgical planning generally use Statistical Shape Models (SSM) or Statistical Shape and Intensity Models (SSIM) for reconstructing bones from X-ray images. However, these algorithms may be slow and depend on definition of the initial conditions, frequently requiring manual intervention during the reconstruction process.

Presently disclosed subject matter describes a system and a method for fast and robust 3D reconstruction of anatomical structures from clinical 2D images such as X-Rays using a full AI-based software pipeline. Reconstruction of a wide variety of anatomical structures, including but not limited to hip, shoulder, and fingers, as well as other bony structures such as spinal vertebrae, may be achieved. Adaptation to non-bony structures such as the biliary system and the coronary arteries, by using X-Ray images with contrast, is also possible. The system and method may be conveniently and robustly adapted to a wide variety of patient anatomies and pathologies by retraining on an appropriate dataset. A model may be trained to separately reconstruct anatomical structures such as the femoral canal, the glenoid vault, and/or anatomical bony landmarks, by including separate segmentations or heat maps of those structures in training data annotations. New pathologies or patient cohorts may be supported by expanding the training set to new examples, and/or artificial data augmentation methods.

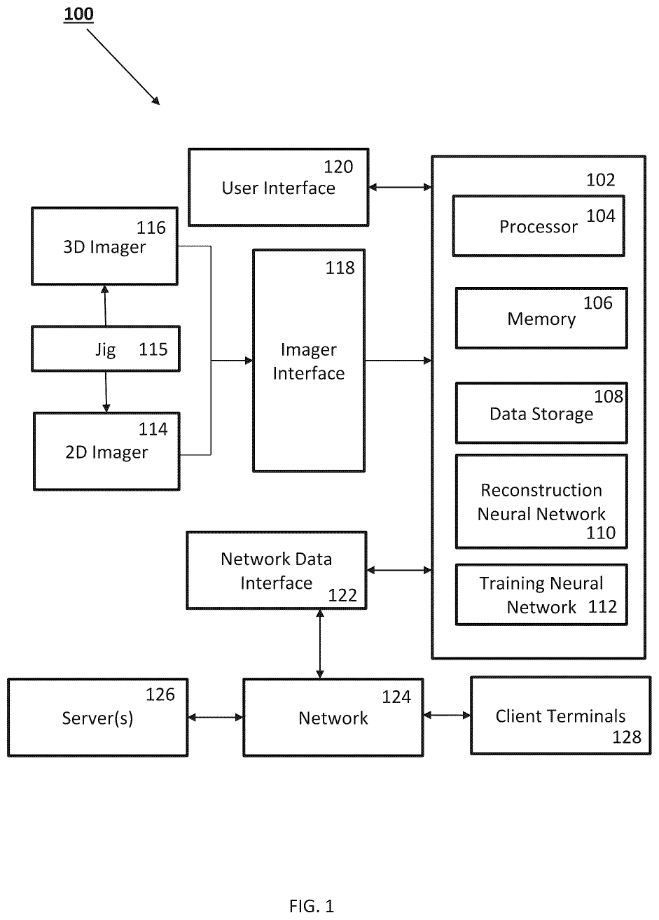

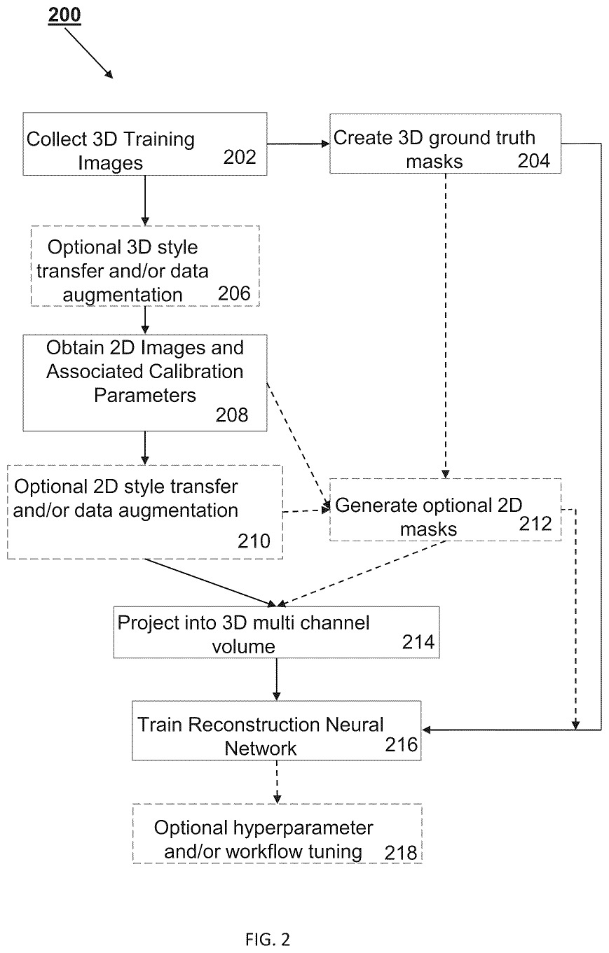

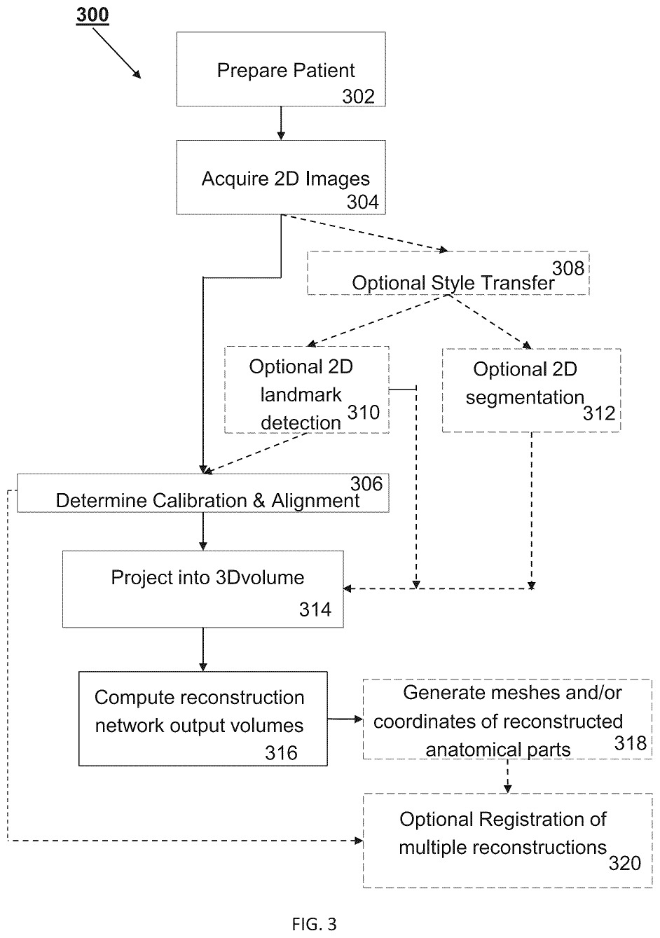

Reference is now made to FIG. 1 which schematically illustrates a block diagram of an exemplary system 100 for AI-based, 3D reconstruction of anatomical images from two-dimensional 2D anatomical images using neural networks, as disclosed further on below. System 100, as shown in the figure and described below, may be used for training a neural network which may be used for the reconstruction process, and may additionally be used for performing the actual reconstruction. Notwithstanding, the skilled person may readily appreciate that system 100, or components therein, may be used only to train the neural network, or alternatively, only to perform the reconstruction. It may be further appreciated, as described further on below, that system 100 may also be used to perform reconstruction of 3D anatomical images from acquired 3D anatomical images. It is noted that system 100 may be used to implement the method shown in FIG. 2 and described further on below for training a neural network, and/or may be used to implement the method shown in FIG. 3 and described further on below for using the trained neural network to reconstruct a 3D anatomical image or images.

System 100 may include a computing device 102 with a processor 104, a memory 106, a data storage 108, a reconstruction neural network 110, and a training neural network 112. System 100 may additionally include a 2D imager 114, a 3D imager 116, an imager interface 118 which may optionally be included in computing device 102, a network data interface 122 which may also be optionally included in the computing device, and a user interface 120. Optionally included in system 100 may also be a network 124 connecting to one or more servers 126 and, additionally or alternatively, to one or more client terminals 128.

In some embodiments, computing device 102 processes 2D anatomical images acquired by 2D imager 114 (e.g., X-ray device, 2D ultrasound device, Fluoroscopic device, etc.) and converts the acquired images to 3D anatomical images. The 2D images may be stored in an image database for example, a PACS server, a cloud storage, a storage server, CD-ROMs, and/or an electronic health record, which may be external to computing device 102 (not shown), or may be internal, for example, integrated in data storage 108. Computing device 102 computes the reconstructed 3D anatomical image from the 2D anatomical image by means of processor 104, which executes aside from system control and operational functions, operations associated with reconstruction neural network 110 and training neural network 112. The operations of training neural network 112 and reconstruction neural network 110 are described further on below with reference to FIG. 2 and FIG. 3, respectively. A training dataset of images created from 3D anatomical images acquired by 3D imaging device 116 (e.g., CT scan, MRI, PET, 3D ultrasound, nuclear imaging, etc.) and corresponding 2D images optionally created from the 3D images may be used by training neural network 110 to generate a trained neural network which may serve as reconstruction neural network 112. The training dataset may be optionally stored in data storage 108. The 3D reconstructed images generated by reconstruction neural network 110 may also be stored in data storage 108, and may be used for planning surgical treatment of a target anatomical structure in a subject. The 3D reconstructed images may also be stored in the training dataset of the 3D images for future training applications.

In some embodiments, training of the neural network, and the application of the trained neural network to 2D anatomical images to compute the reconstructed 3D images, may be implemented by the same computing device 102, and/or by different computing devices 102, for example, one computing device 102 trains the neural network, and transmits the trained neural network to another computing device 102 which uses the trained reconstruction neural network to compute reconstructed 3D image from 2D anatomical images. Computing device 102 receives 2D anatomical images acquired by 2D imaging device 114 for computation of the reconstructed 3D images. Alternatively, or additionally, computing device 102 receives 3D anatomical images acquired by 3D imaging device 116 for creating the training dataset for use by training neural network 112. 2D and/or 3D images may be stored in a data storage, for example data storage 108, which may include for example, a storage server (e.g., PACS server), a computing cloud, virtual memory, and/or a hard disk.

In some embodiments, 2D anatomical images captured by 2D imaging device 114 depict anatomical features and/or anatomical structures within the body of the target subject. 3D anatomical images captured by 3D imaging device 116 may be used to train the neural network for reconstructing 3D images using reconstruction neural network 110 from 2D anatomical images captured by 2D imaging device 114. Exemplary target anatomical structures depicted by 3D anatomical images, which are also depicted by the 3D image reconstructed from the 2D anatomical image may include a hip, a shoulder, and fingers, as well as other bony structures such as spinal vertebrae. Additionally, anatomical structures such as the femoral canal, the glenoid vault, and/or anatomical bony landmarks may also be reconstructed. Optionally, a jig 115 may be used to calibrate the system as described further on below with reference to FIG. 3. Jig 115 may be configured to be positioned over the body area of a subject to be imaged, and may include one or more radio-dense objects such as, for example, ball bearings, or “BBs”, of known size and relative positionings to facilitate calibration.

In some embodiments, computing device 102 may receive the 2D anatomical images and/or 3D anatomical images from imaging device(s) 114 and/or 116 and/or from an external image storage by mean of imager interface 118, which may include for example, a wire connection, a wireless connection, a local bus, a port for connection of a data storage device, a network interface card, other physical interface implementations, and/or virtual interfaces such as, for example, a software interface, a virtual private network (VPN) connection, an application programming interface (API), or a software development kit (SDK)).

In some embodiments, hardware processor 104 may be implemented, for example, as a central processing unit (CPU), a graphics processing unit(s) (GPU), a field programmable gate array (FPGA), a digital signal processor (DSP), and/or application specific integrated circuit (ASIC). Processor 104 may include one or more processors which may be arranged for parallel processing, as clusters and/or as one or more multi core processing units. Memory 106 stores code instruction for execution by hardware processor 102, for example, a random access memory (RAM), a read-only memory (ROM), and/or a storage device, for example, non-volatile memory, magnetic media, semiconductor memory devices, hard drive, removable storage, and optical media such as a DVD or a CD-ROM.

In some embodiments, data storage device 108 may store data, for example, a trained dataset of 2D images and/or 3D images, and optionally a reconstruction dataset that stores the reconstructed 3D images and/or computed 3D images for future use in the training dataset. Data storage 108 may be implemented, for example as a memory, a local hard-drive, a removable storage device, an optical disk, a storage device, and/or as a remote server and/or computing cloud which may be accessed, for example, over network 124. It is noted that code may be stored in data storage 108, with executing portions loaded into memory 106 for execution by processor 104. It is further noted that data storage 108, although shown in FIG. 1 as being internally located in computing device 102, it may be wholly or partially located external to the computing device.

In some embodiments, computing device 102 may include data interface 122, optionally a network interface, for connecting to network 124. Data interface 122 may include, for example, one or more of, a network interface card, a wireless interface to connect to a wireless network, a physical interface for connecting to a cable for network connectivity, a virtual interface implemented in software, network communication software providing higher layers of network connectivity, and/or other implementations. Computing device 102 may access one or more remote servers 126 using network 124, for example, to download updated training datasets, to download components for inclusion in the training datasets (e.g., 3D anatomical images) and/or to download updated versions of operational code, training code, reconstruction code, reconstruction neural network 110, and/or training neural network 112.

In some embodiments, imager interface 118 and data interface 122 may be implemented as a single interface, for example, as a network interface or a single software interface, and/or as two independent software interfaces such as, for examples, APIs and network ports. Additionally, or alternatively, they may be implemented as hardware interfaces, for example, two network interfaces, and/or as a combination of hardware and software interfaces, for example, a single network interface and two software interfaces, two virtual interfaces on a common physical interface, or virtual networks on a common network port.

In some embodiments, computing device 102 may communicate using network 124 with one or more of client terminals 128, for example, when computing device 102 acts as a server that computes the reconstructed 3D image from a provided 2D anatomical image. Additionally, or alternatively, computing device 102 may use a communication channel such as a direct link that includes a cable or wireless, and/or an indirect link that includes an intermediary computing device such as a server and/or a storage device, Client terminal 128 may provide the 2D anatomical image and receive the reconstructed 3D image computed by computing device 102. The obtained reconstructed 3D image may be, for example, presented within a viewing application for viewing (e.g., by a radiologist) on a display of the client terminal, and/or fed into a surgical planning application (and/or other application such as CAD) installed on client terminals 128.

In some embodiments, server 126 may be implemented as an image server in the external data storage, for example, a PACS server, or other. Server 126 may store new 2D anatomical images as they are captured, and/or may store 3D anatomical images used for creating training datasets. In another implementation, server 126 may be in communication with the image server and computing device 102. Server 126 may coordinate between the image server and computing device 102, for example, transmitting newly received 2D anatomical images from server 126 to computing device 102 for computation of the reconstructed 3D image. Optionally, server 126 may perform one or more features described with reference to client terminals 128, for example, feeding the reconstructed 3D image into surgical planning applications (which may be locally installed on server 126).

In some embodiments, computing device 102 and/or client terminals 128 and/or server 126 include or are in communication with a user interface 120 that includes a mechanism designed for a user to enter data, for example, to select target anatomical structures for computation of the reconstructed 3D image, select surgical planning applications for execution, and/or view the obtained 2D images and/or view the reconstructed 3D images. Exemplary user interfaces 120 include, for example, one or more of, a touchscreen, a display, a keyboard, a mouse, and voice activated software using speakers and microphone.

In some embodiments, computing device 102 may be implemented as, for example, a client terminal, a server, a virtual server, a radiology workstation, a surgical planning workstation, a virtual machine, a computing cloud, a mobile device, a desktop computer, a thin client, a smartphone, a tablet computer, a laptop computer, a wearable computer, glasses computer, and a watch computer. Computing device 102 may include a visualization workstation that may be added to a radiology workstation and/or surgical workstation and/or other devices for enabling the user to view the reconstructed 3D images and/or plan surgery and/or other treatments using the reconstructed 3D images.

In some embodiments, computing device 102 may act as one or more servers, for example, a network server, web server, a computing cloud, a virtual server, and may provide functions associated with the operation of training neural network 110 and/or reconstruction neural network 112 to one or more client terminals 128. Client terminals 128 may be used by a user for viewing anatomical images, for running surgical planning applications including planning surgery using the reconstructed 3D images, for running computer aided diagnosis (CAD) applications for automated analysis of the reconstructed 3D images, as remotely located radiology workstations, as remote picture archiving and communication system (PACS) server, or as a remote electronic medical record (EMR) server over a network 124. Client terminals 108 may be implemented as, for example, a surgical planning workstation, a radiology workstation, a desktop computer, or a mobile device such as, for example, a laptop, a smartphone, glasses, a wearable device, among other mobile devices.

Reference is now made to FIG. 2 which illustrates an exemplary flow chart 200 of the training operation of training neural network 112. Once trained, training neural network 112 may serve to perform reconstruction of 3D anatomical images from 2D images as described in FIG. 3 with reference to reconstruction neural network 110 and/or may serve to train other neural networks to serve as reconstruction neural networks. In describing the operation of training neural network 112, reference may be made to system 100 and components therein.

In the steps described below, code associated with the operation of training neural network 112 may be executed by processor 104. Data storage, as required during execution of each step of the operation of training neural network 112 may include the use of data storage 108 (optionally including the image database). It is noted that some of the steps are optional and the blocks in the flowchart shown in FIG. 2 associated with optional steps are indicated by hatched borders. Arrows in the flow chart with hatched lines are indicative of an optional path in the task flow.

At 202, a training dataset of 3D images may be collected. The 3D images may include scans acquired from 3D imagers such as, for example, 3D imager 116 which may include an MRI device, a CT device, a 3D ultrasound device, a PET device, a nuclear imaging device, among other types of 3D imagers, and may have been collected from subjects who have undergone or may undergo a similar (or same) reconstruction process. The 3D images may optionally include reconstructed 3D images from 2D images generated by computing device 102, and/or 3D images which may be commercially obtained as training datasets. The 3D images may include anatomical parts to be reconstructed and derived from subjects and pathologies representative of the reconstruction case, for example, knee CT images of joint replacement patients with osteoarthritis.

At 204, a 3D ground truth mask may be created for each 3D image in the training dataset. The 3D mask may be created for an entire bone, and/or separate masks for bone cortex and for internal structures such as femoral canal or glenoid vault, and/or for anatomical landmarks or interest points. The masks may be created by manual annotation, for example by means of user interface 120, and/or by running a 3D segmentation algorithm, for example, using a neural network such as UNet or similar architecture.

At 206, 3D style transfer and/or data augmentation may be optionally performed on the training dataset from Step 202. A 3D style transfer algorithm such as, for example, Cycle-GAN, may be applied to 3D images in the training dataset that require a change in modality to resemble more closely that of X-rays. For example, MRI images may be transformed to CT-type images. Augmentation may be performed to 3D images in the training dataset in order to increase variability in the 3D images, for example, by adding perturbations, noise, and/or other variability (augmentation) parameters. The augmentation may be performed using a 3D augmentation algorithm such as, for example affine transformations, elastic transformations, blur, gaussian noise, GAN-based augmentation etc. Optionally, the 3D images which are to be augmented may be replicated and the augmentation performed on the replica.

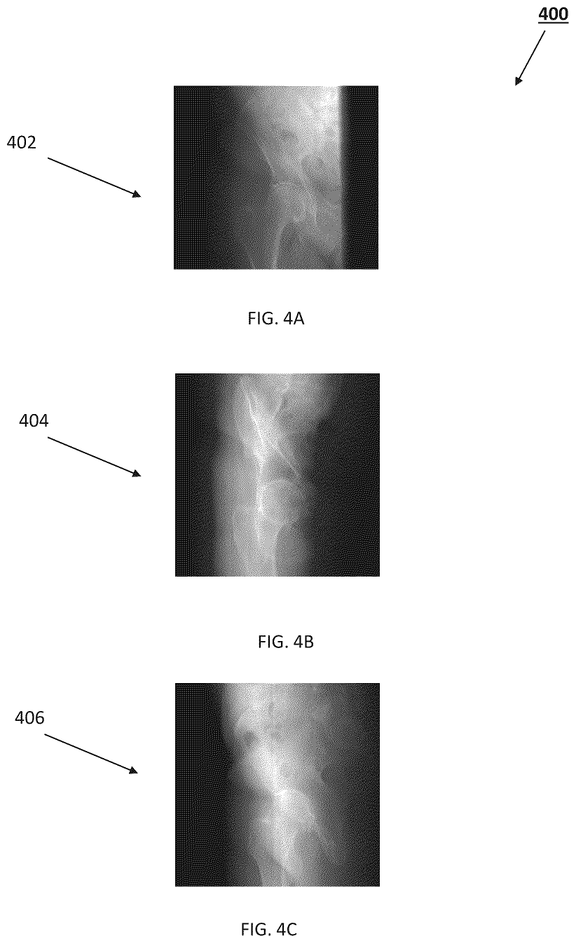

At 208, 2D images and their associated calibration parameters may be obtained directly from 2D imaging devices, for example, 2D imager 114, and/or by generating Digital Reconstructed Radiographs (DRRs) and simulating the expected distribution of calibration parameters. The one or more (DRRs) may be generated from the 3D images in the training dataset from Step 202, and from those images which have optionally been subject to style transfer and/or augmentation in Step 206. The DRRs may be generated to include views having a predefined set of angles that match the planned angles of the input X-rays. For example, for some cases of knee reconstruction, anterior-posterior (AP) and lateral angles may be used. As another example, in some cases of hip joint reconstruction, the AP angle and two oblique views at +45 degrees and −45 degrees may be used. Reference is also made to FIG. 4A, 4B, and 4C, which show examples of a set of 3 DRRs of a hip joint 400 generated in the step, DRR 402 from AP (FIG. 4A), DRR 404 from +45 degrees (FIG. 4B), and DRR 406 from −45 degrees angles (FIG. 4C). Optionally, each DRR angle may be slightly varied in order to simulate expected deviations of the actual input X-ray angles from their predetermined values. Optionally, the angle between multiple anatomical parts may be varied between the views in order to simulate joint articulation.

At 210, 2D style transfer and/or data augmentation may be optionally performed on the DRRs from Step 208. A 2D style transfer algorithm such as, for example, Cycle-GAN, may be applied to the DRRs to change their modality to resemble more closely that of X-rays. Augmentation may be performed on the DRRs in order to increase variability in the DRRs, for example, by adding perturbations, noise, and/or other variability (augmentation) parameters. The augmentation may be performed using a 2D augmentation algorithm such as, for example affine transformations, projective transformations, elastic transformations, blur, gaussian noise, GAN-based augmentation etc. Optionally, the DRRs which are to be augmented may be replicated and the augmentation performed on the replica.

At 212, for the desired anatomical areas which will be subject to reconstruction in a subject, 2D masks may be optionally generated from the DRRs from Step 208, and optionally from the DRRs which have optionally been subject to style transfer and/or augmentation in Step 210. Optionally, 2D masks may also be generated from the 3D masks from Step 204. To generate the 2D masks, a 2D segmentation neural network trained to perform the masking may be used, for example, UNet or similar architecture. The neural network may be trained separately from reconstruction neural network 110, or alternatively, may be trained jointly with the reconstruction neural network. Additionally, or alternatively, the 2D masks may be created by manual annotation, for example by means of user interface 120. Optionally, perturbations, noise, and/or other variability (augmentation) parameters may be introduced into the 2D masks for augmentation purposes.

At 214, the DRRs from Steps 208 and/or the 2D masks from optional Steps 210 and 212, may be projected into a 3D multi-channel volume which may serve as an input to reconstruction neural network 110. The 3D multi-channel volume, which may be shaped as a cube, is formed by a 3D array of voxels with each cube channel corresponding to a 2D image (a DRR). The 3D volume may be formed considering expected internal calibration parameters of 2D imager 114 and associated projection matrices, and the angles between the views and the anatomical points that were used to generate the DRRs. Optionally, jitter may be introduced to the calibration parameters and/or angles in order to simulate expected calibration errors and develop robustness to these errors in reconstruction neural network 110. Multiple 2D input channels (images) may be used for each view, and may include original 2D images, DRRs including those optionally after style transfer and/or augmentation, 2D masks, heat maps of landmarks, and/or other feature representations of a 2D input. Each 2D input channel may be projected into the 3D multi-channel volume channel using a suitable transformation matrix and suitable interpolation methods. For example, a projective transformation may be used and represented by a 3×4 camera calibration matrix in a homogeneous coordinate system. Each point in the 3D volume may be represented by homogeneous real world coordinates r=[x,y,z,1] and voxel indices [i,j,k], and projected into the 2D space using camera calibration matrix P by equation: [w*n, w*m, w]=P*r, where w is an arbitrary scaling factor that is divided out when converting to the non-homogeneous pixel indices [n,m]. The specified channel C is then filled with values from the 2D image I using C[i,j,k]=I[n,m]. Optionally, a nearest-neighbours interpolation method may be used. Additionally or alternatively, 3D linear interpolation may be used.

At 216, reconstruction neural network 110, which may include, for example, UNet or a similar neural network architecture, may be trained to restore the 3D masks of each anatomical structure to be reconstructed based on the 3D multi-channel volumes projected in Step 214. The 3D ground truth masks from Step 204 may be used in training reconstruction neural network 110. Optionally, a loss function may include a Dice coefficient term to enforce consistency of 3D output masks with the 3D ground truth masks. Optionally, a voxel wise cross-entropy loss with a spatial weight to enhance near surface voxels (Supervised loss):

$${loss}_{C E} = - \frac{1}{N} {\sum }_{i = 1}^{N} {\sum }_{k = 0}^{K} D W M ( i ) \cdot {q}_{k} ( i ) \cdot log log ( {p}_{k} ( i ) )$$

i—the index of a voxel
N—the total number of voxels
k—the class label (for example, bone, osteophytes or background)
K—number of classes
qₖ(i)∈{0, 1}—ground truth
Pₖ(i)∈(0, 1)—prediction probabilities,
DWM: distance weighting matrix, which may be for example defined as follows:

DWM(i)=1+γ·exp(−d(i)/σ)

d—distance from anatomical structure surface
γ, σ—constants Optionally, to preserve precise structures seen in the 2D images, a projection consistency loss function may be used to encourage the network to conform to the 2D input X-Rays or segmentation masks. For example, the projection consistency loss function may consist of an unsupervised reconstruction loss to align the neural network prediction of anatomical structure's probability map with the input X-ray images. In the case of inputs consisting of AP and lateral views, this unsupervised reconstruction loss may be defined as follows:

$${Loss}_{r e c o n s t} = 1 - \frac{1}{2} ( NGCC ( {I}_{L a t} , {DRR}_{L a t} ) + N GCC ( {I}_{A P} , {DRR}_{A P} ) )$$

where NGCC is the Normalized Gradient Cross Correlation, I_{Lat}, I_{AP} are the input X-ray images from AP and lateral views respectively, and DRR_{Lat}, DRR_{AP} are DRRs applied on the maximum over the channels of the network prediction.

At 218, multiple training sessions of repeated runs of Steps 202 to 216 may optionally be compared in order to tune hyperparameters and workflow. The results of the multiple training sessions may be compared, for example, by measuring the Dice coefficient between the 3D ground truth masks and the 3D output masks on a validation dataset. The comparison may be used to determine the optimal angles for a specific anatomical area, and/or to measure sensitivity of the system to angular deviations and image quality issues. Tuning the hyperparameters and the workflow may include controlling the angles at which the DRRs are generated, employing augmentations, and measuring the reconstruction performance under various conditions.

Reference is now made to FIG. 3 which illustrates an exemplary flow chart 300 of a 3D anatomical reconstruction operation using reconstruction neural network 110. Reconstruction neural network 110 may serve to perform reconstruction of 3D anatomical images from 2D images following training by training neural network 110. Reconstruction neural network 110 may be the same neural network as training neural network 112 following a training operation, or alternatively, may be a different neural network trained by training neural network 112. In describing the operation of reconstruction neural network 100, reference may again be made to system 100 and components therein.

In the steps described below, code associated with the operation of reconstruction neural network 110 may be executed by processor 104. Data storage, as required during execution of each step of the operation of reconstruction neural network 112 may include the use of data storage 108. It is noted that some of the steps are optional and the blocks in the flowchart shown in FIG. 3 associated with optional steps are indicated by hatched borders. Arrows in the flow chart with hatched lines are indicative of an optional path in the workflow.

At 302, a subject who is going to undergo an anatomical reconstruction process may be prepared by affixing a calibration jig, for example calibration jig 115, proximally to the anatomical part to be reconstructed. Jig 115 may be affixed prior to the subject being exposed to 2D imaging using a 2D imager, for example 2D imager 114 which may include an X-ray device, a 2D ultrasound device, a Fluoroscopic device, or other suitable 2D anatomical imaging device. Jig 115 may be affixed while the subject is positioned on an auxiliary jig placement device prior to being positioned on 2D imager 114, or alternatively while the subject is positioned on the 2D imager device. Where multiple jigs 115 are required, these may be affixed to the subject while positioned on the auxiliary jig placement device and/or while positioned on 2D imager 114. Optionally, multiple jigs may be affixed to different anatomical parts prior to, or during 2D image acquisition, to assist in subsequent correction for articulation of joints between multiple angle image acquisitions. For convenience hereinafter, the 2D image or images may also be referred to as X-rays. The subject may be positioned on 2D imager 114 at a predetermined angle, or angles, to allow jig 115 to be within the field of view of the imager. The predetermined angles may be similar to the view angles used in training neural network 112, and should provide maximum visibility of jig 115 and minimal occlusion of the bone. For example, if the 2D images are to be acquired at 0 and 90 degrees, jig 115 may be positioned at approximately 45 degrees.

At 304, 2D images, for example one or more X-rays, are acquired of the reconstruction area using 2D imager 114. Optionally, images of multiple areas, such as, for example, the knee, thigh, hip, and ankle, may be acquired with a common jig in view in order to allow for subsequent registration between reconstructions of multiple areas. At 306, the calibration parameters may be determined. The calibration parameters may be determined by any one of, or any combination of, the following: jig detection, landmark detection, and calibration parameters provided by the 2D imager during image acquisition and/or provided by the system or determined ahead of time by separately imaging a jig. For jig calibration purposes, jig 115 is located in the acquired 2D images and the locations (coordinates) of individual steps on the jig are identified, for example using landmark detection neural networks and/or template matching method and/or other suitable methods. The individual steps may be identified by means of ball bearings, BBs, or other suitable radio dense objects used in jig 115 which may be identified in the 2D images.

The optional landmark coordinates from step 310, may be used to compute internal and external calibration parameters to be applied to the 2D images. These parameters may include focal length, principal point, 6 degree-of-freedom (DOF) transformation matrices between multiple X-ray views, and/or 6-DOF transformation matrices between individual bones in order to correct for articulation of the joint(s) between multiple view image acquisitions. The calibration parameters may be computed using adjustment algorithms such as, for example, the P3P or bundle adjustment algorithm, considering the known structure of jig 115. Additionally, pairing of specific landmark points in the multiple views as given by the output of the landmark detection algorithm in optional Step 310 may be considered.

At 308, 2D style transfer may be optionally performed on the acquired 2D images from Step 304. A 2D style transfer algorithm such as, for example, neural network Cycle-GAN, may be applied to the 2D images to change their modality to resemble the DRR format used in training neural network. Optionally, acquired X-ray images may be converted to DRR images in order to resemble DRR images from CT scans.

At 310, anatomical landmarks may be optionally detected in the DRR images from Step 308, for example by using a neural network trained to perform landmark detections, for example, multi-channel UNet or similar architecture. The landmark detection algorithm may be constructed to provide pairings of specific landmarks across the multiple angle views of the acquired 2D images in Step 302.

At 312, 2D segmentation may be optionally performed on the DRRs from Step 308, with segmentation masks computed for each bone depending on whether or not 2D segmentation was used in training neural network 112. The segmentation masks may be generated using a segmentation neural network, for example, UNet or similar architecture, each bone optionally represented by one or more segmentation masks, and separate segmentation masks used to represent the bone cortex and/or internal structures such as the femoral canal or glenoid vault.

At 314, based on the internal and external calibration parameters and associated projection matrices from Step 306, each 2D image may be projected into a 3D input volume. Multiple 2D input channels (images) may be used for each view, and may include original 2D images, DRRs after style transfer, 2D segmentation masks, heat maps of landmarks, and/or other feature representations of a 2D input. Each 2D input channel may be projected into separate 3D input volume channels using a suitable transformation matrix and suitable interpolation methods, similar, for example, as previously described for Step 214.

At 316, a 3D reconstruction output volume is computed based on the projected input volume. The reconstruction neural network 110 that was trained in step 216 may be used to restore the 3D segmentation masks and/or heat maps of each desired anatomical structure. The 3D segmentation masks may represent the entire bone, and/or separate masks for bone cortex and for internal structures such as femoral canal or glenoid vault, and/or other anatomical interest points such as landmarks.

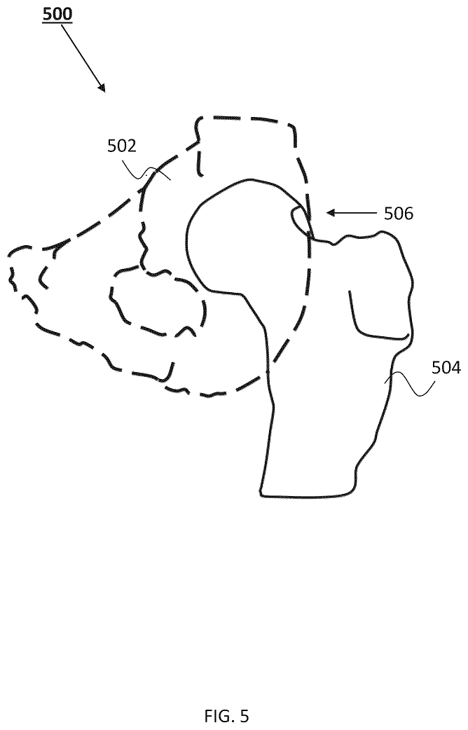

At 318, the 3D segmentation masks from Step 316 associated with the anatomical structures may be optionally converted to meshes using polygonal mesh extraction techniques such as, for example, Marching Cubes. Regarding the optional reconstructing landmark points also from Step 316, the heat map may be post-processed, for example by peak detection, to obtain the landmark coordinates. Reference is also made to FIG. 5 which shows an exemplary representation of a 3D reconstruction output volume 500 generated in the step. Shown therein is a representation of mesh models of a portion of a pelvis 502, a femur 504, and the joint 506 connecting the femur and the pelvis.

At 320, optional registration of multiple reconstructions may be performed. Repeated runs of Steps 302 -320 may be used to generate multiple reconstructions of a number of anatomical areas of the same subject, from multiple image sets. For example, an image set of one or more views may be acquired for the knee, another for the hip or specific landmarks therein, and another for the ankle or specific landmarks therein. By placing calibration jig 115 (or jigs) in view in two or more of the image sets, 3D transformation between the multiple reconstructions may be determined. Optionally, multiple views may be used for each area imaged. Alternatively, a single view may be used for some of the areas, and a 3D transformation may be determined based on the known jig structure using mathematical methods such as, for example, [solving a system of linear equations for the calibration matrix using Singular Value Decomposition. This registration of multiple reconstructed areas may support 3D surgical planning, for example, by determining the anatomical axes of an entire leg based on femur and ankle 3D landmarks when planning and selecting a knee replacement implant based on knee joint bones reconstruction.

While this disclosure has been described in terms of certain embodiments and generally associated methods, alterations and permutations of the embodiments and methods will be apparent to those skilled in the art. The disclosure is to be understood as not limited by the specific embodiments described herein, but only by the scope of the appended claims.

All references mentioned in this specification are herein incorporated in their entirety by reference into the specification, to the same extent as if each individual reference was specifically and individually indicated to be incorporated herein by reference. In addition, citation or identification of any reference in this application shall not be construed as an admission that such reference is available as prior art to the present application.

1-37. (canceled)

38. A medical imaging method, comprising:
generating a three-dimensional (3D) volume from a two-dimensional (2D) image of an anatomical structure and an associated calibration parameter of the 2D image; and
generating a reconstructed 3D mask and/or a heat map representing a shape and/or feature of the anatomical structure directly from the 3D volume using a deep neural network (DNN).

39. The method of claim 38, wherein the 2D image is selected from the group consisting of a 2D image from a 2D imager, a digital reconstructed radiograph (DRR), a 2D segmentation mask, a heat map of a landmark, and a feature representation of a 2D image.

40. The method of claim 38, wherein the generating a 3D volume includes generating a single 3D volume using a direct projection function.

41. The method of claim 38, further comprising generating two or more 3D volumes from two or more 2D images and their associated calibration parameters, wherein the generating of the two or more 3D volumes includes applying a direct projection function separately to each of the two or more 2D images.

42. The method of claim 38, wherein the DNN is trained using as ground truth output a 3D mask and/or a heat map generated by segmenting or annotating features in a computerized tomography (CT) scan from a training dataset, and using a 2D DRR and/or 2D segmentation mask and/or 2D heat map as input to the training.

43. The method of claim 38, wherein the DNN is trained using as ground truth output a 3D mask and/or a heat map generated by segmenting or annotating features in 3D images acquired from an imaging device that is not a computerized tomography (CT) device, the imaging device selected from the group consisting of a magnetic resonance imaging (MRI) device, a 3D ultrasound device, a positron emission tomography (PET) device, and a nuclear imaging device.

44. The method of claim 43, further comprising applying a 3D style transfer algorithm to the 3D images to enable conversion of the 3D images to resemble X-rays for use in training the DNN.

45. The method of claim 38, wherein the generating a reconstructed 3D mask and/or a heat map is performed without generating or using a 3D computerized tomography volume.

46. The method of claim 38, wherein the associated calibration parameter is determined by detecting a calibration jig and/or anatomical landmarks in two or more 2D images.

47. The method of claim 38, wherein the associated calibration parameter is determined using an automatic landmark detection algorithm such as a neural network.

48. The method of claim 38, wherein the reconstructed 3D mask represents an entire bone and/or another anatomical interest point such as an anatomical landmark.

49. The method of claim 38, wherein the reconstructed 3D mask includes a plurality of separate 3D masks for bone cortex and for internal bone structures.

50. The method of claim 38, wherein the reconstructed 3D heat map represents an entire bone and/or another anatomical interest point such as an anatomical landmark.

51. A system for reconstructing a three-dimensional (3D) image of an anatomical structure from a two-dimensional (2D) image comprising:
a storage device including a 2D image of an anatomical structure and a calibration parameter associated with the 2D image;
a processor; and
a memory storing instructions executable by the processor and which cause the system to perform the following steps:
generating a 3D volume from the 2D image and the associated calibration parameter of the 2D image; and
generating a reconstructed 3D mask and/or heat map representing a shape and/or feature of the anatomical structure directly from the 3D volume using a deep neural network (DNN).

52. The system of claim 51, wherein the 2D image is selected from the group consisting of a 2D image from a 2D imager, a digital reconstructed radiograph (DRR), a 2D segmentation mask, a heat map of a landmark, and a feature representation of a 2D image.

53. The system of claim 51, wherein the generating a 3D volume includes generating a single 3D volume using a direct projection function.

54. The system of claim 51, wherein the instructions executable by the processor cause the system to further perform generating two or more 3D volumes from two or more 2D images and their associated calibration parameters, and wherein the generating of the two or more 3D volumes includes applying a direct projection function separately to each of the two or more 2D images.

55. The system of claim 51, wherein the storage device further includes a training dataset, wherein the DNN is trained using as ground truth output a 3D mask and/or a heat map generated by segmenting or annotating features in a computerized tomography scan from the training dataset, and using a 2D DRR and/or 2D segmentation mask and/or 2D heat map as input to the training.

56. The system of claim 51, wherein the storage device further includes 3D images acquired from an imaging device that is not a computerized tomography (CT) device, the imaging device selected from the group consisting of a magnetic resonance imaging (MRI) device, a 3D ultrasound device, a positron emission tomography (PET) device, and a nuclear imaging device, and wherein the DNN is trained using as ground truth output a 3D mask and/or heat map generated by segmenting or annotating features in the 3D images.

57. The system of claim 56, wherein a 3D style transfer algorithm is applied to the 3D images to enable conversion of the 3D images to resemble X-rays for use in training the DNN.

58. The system of claim 51, wherein the generating a reconstructed 3D mask and/or a heat map is performed without generating or using a 3D computerized tomography volume.

59. The system of claim 51, wherein the associated calibration parameter is determined by detecting a calibration jig and/or anatomical landmarks in two or more 2D images.

60. The system of claim 51, wherein the associated calibration parameter is determined using an automatic landmark detection algorithm such as a neural network.

61. The system of claim 51, wherein the reconstructed 3D mask represents an entire bone and/or another anatomical interest point such as an anatomical landmark.

62. The system of claim 51, wherein the reconstructed 3D mask includes a plurality of separate 3D masks for bone cortex and for internal bone structures.

63. The system of claim 51, wherein the reconstructed 3D heat map represents an entire bone and/or another anatomical interest point such as an anatomical landmark.
